(19) United States
(12) Patent Application Publication
(10) Pub. No.: US 2003/0049337 A1
(43) Pub. Date: Mar. 13, 2003

(54) Composition for preventing fructose absorption

(75) Inventors: Keiichiro Sugimoto, Osaka (JP); Kazuya Nakagawa, Osaka (JP); Saburo Konoeda, Osaka (JP); Syuichi Hayashi, Osaka (JP); Hiroshi Inui, Osaka (JP); Yoshihisa Nakano, Osaka (JP)

Correspondence Address: SUGHRUE MION, PLLC, 2100 Pennsylvania Avenue, N.W., Washington, DC, 20037-3213, US

(73) Assignee: NAGAOKA PERFUMERY CO., LTD

(21) Appl. No.: 10/133,455

(22) Filed: Apr. 29, 2002

(30) Foreign Application Priority Data
Sep. 11, 2001 (JP) 2001/275572
Dec. 4, 2001 (JP) 2001/370585

(51) Int. Cl. A61K 35/78; A61K 31/7012

(52) U.S. Cl. 424/742; 514/53

(57) ABSTRACT

There is provided a composition for preventing fructose absorption, which contains an extract of a plant of the genus eucalyptus as an active ingredient. This extract does not prevent absorption of the glucose from intestines, but prevents absorption of fructose specifically, and therefore is useful in food, drugs, etc. Moreover, there is provided a composition for prevention or treatment of hyperlipidemia and fatty liver and for inhibition of accumulation of internal-organs fat, which contains an extract of a plant of the genus eucalyptus as an active ingredient.

### FIELD OF THE INVENTION

The present invention relates to a composition for preventing fructose absorption, which contains an extract extracted from a plant of the genus eucalyptus as an active ingredient, and a method of preventing fructose absorption. In more detail, the present invention relates to a composition for preventing fructose absorption, which can prevent absorption of fructose in the living body specifically, and is effective in prevention or medical treatment of hyperlipemia and fatty liver, and inhibition and amelioration of accumulation of internal-organs fat, and a method of preventing fructose absorption.

### BACKGROUND OF THE INVENTION

Obesity is in the state which fat accumulated superfluously inside of the body, and one of the causes which fat accumulates inside of the body is superfluous ingestion of saccharides (carbohydrates). Generally, when carbohydrate contained in ingesta is taken in inside of the body, it will be digested with a digestive enzyme, mainly serves as monosaccharide, and is absorbed by the inside of the body from an intestinal tract. Monosaccharide is transformed into fat in a fat cell, and a fat tissue is formed.

Glucose (grape sugar) which is a kind of monosaccharide is metabolized after digestive absorption by the enzyme group called as glycolysis. Transformation to fat is controlled by undergoing metabolism regulation in the stage of passing through phosphofructokinase among these enzymes. However, since fructose (fruit sugar) which is the same monosaccharide as glucose is metabolized by the system which does not undergo metabolic regulation through a different metabolic pathway from glucose, it is transformed into fat easily into liver, and obesity tends to rise.

Although liver changes generated fat into a very low-density lipoprotein (VLDL) and emits it into serum, when the lipoprotein production ability of liver stops catching up, fat is accumulated into liver and is advanced from fatty liver to liver cirrhosis. On the other hand, an increase of VLDL secretion causes hyperlipemia and hypercholesterolemia. Moreover, also from producing the fall of insulin susceptibility (insulin resistance), superfluous ingestion of carbohydrate causes hyperlipidemia and worsens it. The increase in the VLDL concentration in blood is the main cause of arteriosclerosis. Arteriosclerosis causes thrombus etc. and has become one of the factors of various circulatory organ system diseases, such as cerebral infarction and myocardial infarction. If insulin resistance rises, it comes to cause diabetes, high blood pressure, etc. besides the above diseases. Furthermore, since extensive ingestion of fructose promotes an uric acid synthesis, it worsens hyperuricemia (gout is started if hyperuricemia goes on).

Sugar (sucrose) which is a kind of carbohydrates is decomposed into glucose and fructose by the carbohydrates-decomposition digestive enzyme. When sugar is taken in large quantities, the blood sugar level goes abruptly up by prompt absorption of glucose, and in connection with it, insulin is also secreted at once. Since insulin has the work which activates the system transformed from fructose or glucose to lipid; a glycogen synthesis; and taking in glucose to a fat cell, the case of taking in sugar tends to accelerate obesity, as compared with the case where glucose or fructose is taken in independently. Therefore, sugar are attached importance to on medicine as obesity cause substance, rather than glucose, fructose and starch.

For this reason, various alternative sweeteners, such as a natural extract, e.g. the glycyrrhizac radix extract which uses glycyrrhizinic acid as a sweet taste ingredient, the stevia extract which uses steviol glycoside as a sweet taste ingredient, RAKANKA (Momovdiae grosvenori)extract which uses mogrosides as a sweet taste ingredient, sugar alcohol (xylitol, erythritol, sorbitol, etc.), non-saccharide system synthesis sweetener (aspartame, sucralose, etc.), are developed. However, since sugar is most excellent on flavor as a sweetener in spite of it, the importance of sugar in a sweetener does not change at all, and sugar is consumed in large quantities by juice, confectionery, cooking, etc.

Accordingly, since it is most difficult to avoid ingestion of the food and drink containing sugar, it is thought to study the method which cannot be growing fat easily, even if sugar are taken in in large quantities. Then, study has been energetically carried out about the two following methods.

(1) A method for delaying absorption of saccharide in the living body by preventing a carbohydrate-decomposition digestive enzyme.

(2) A method for making absorption of monosaccharide in an intestinal tract prevent.

Among these, as for the method of (1), many use of α-amylase inhibitor or β-glucosidase inhibitor etc. is reported until now. For example, they are acarbose (Japanese Patent Publication No. 54-39474), tea catechin (Japanese Unexamined Patent Publication No. 3-133928), and guava (Japanese Unexamined Patent Publication No. 7-59539), etc. However, prevention of the carbohydrate-decomposition digestive enzyme does not necessarily prevent absorption of carbohydrate, and has only the effect of delaying digestion.

Therefore, because the method of the above (1) controls the rapid rise of the after-a-meal blood sugar level, there is an effect which stabilizes insulin secretion. However, according to the method of (1), since the ingestion total amount of calories does not change, it should be said that the anti-obesity effect is inadequate. Moreover, although monosaccharide may be added as raw material in a processed food, when oral ingestion of monosaccharide is carried out, the activity prevention effect of the carbohydrate-decomposition digestive enzyme cannot be expected at all.

On the other hand, the method of (2) can expect the effect of reducing the ingestion total amount of calories, if the absorption of glucose and fructose generated by digestion of sugar can be prevented.

In connection with glucose, the substance which prevents the absorption of glucose in an intestinal tract has been studied briskly. As such an absorption prevention substance, phlorizin (D. F.Diedrich,Biochem.Biophys.Acta,71, 688(1963)), and phloretin (D. F.Diedrich,Biochem.Biophys.,117,248(1966)) are known for many years, and Gymnema(Yoshioka, J. Yonago Med Ass, 37 and 142, (1986), and Hiji et al., Food Style 21, 3, 58 (1999)), brown sugar (Matsuura et al., J. Trad Med., 7,446 (1990)), etc. are known recently. However, glucose is the most important monosaccharide on the biochemistry of a mammal, and is the main energy source of various organizations. Especially the energy source of a brain is usually glucose only. Consequently, preventing absorption of glucose powerfully has a problem on safe.

On the other hand, fructose is hardly confirmed other than a role of a calorie source, although various diseases including obesity are caused by the extensive ingestion as described above. For this reason, it is thought that the nutritional importance of fructose is lower than glucose. Therefore, it can be said that it is the best method to make absorption of fructose through an intestinal tract in the living body prevent specifically, as the obesity prevention method in the case of taking in carbohydrate superfluously.

However, although many substances derived from the natural substance, such as Gymnema, are reported in connection with prevention of the glucose absorption in the living body as described above, the substance derived from the natural substance which makes absorption of fructose in the living body prevent specifically is hardly known.

### SUMMARY OF THE INVENTION

A main object of the present invention is to provide a composition for preventing absorption of fructose in the living body specifically, and having high safety.

Another object of the present invention is to provide a composition for preventing or treating hyperlipemia and fatty liver, and for inhibiting or improving the accumulation of internal-organs fat.

It is further object of the present invention to provide a method of safely preventing fructose absorption in the living body specifically by the substance derived from the natural substance.

The present inventors advanced research intensively for the extract of various plants, in order to provide a composition for preventing absorption of fructose, derived from the natural substance having high safety. As a result, although it was found out that a eucalyptus has the prevention activity (IC₅₀=2.66 mg/mL) to sucrase which is a kind of a carbohydrate-decomposition digestive enzyme, when compared with catechin (IC₅₀=1.61 mg/mL), for example, it was thought that prevention activity of the eucalyptus was not so strong, therefore the action which controls the increase in weight was hardly showed.

However, when the test for checking the absorption of glucose and fructose in an intestinal tract was conducted, the following surprising facts were found out. That is, although a eucalyptus extract does not prevent absorption of glucose from an intestinal tract in the living body at all, it prevents absorption of fructose powerfully. The present invention was completed by this.

Hence, a composition for preventing absorption of fructose of the present invention comprises (1) a biologically effective amount of an extract extracted from a plant of the genus eucalyptus as an activity ingredient, and (2) a biologically acceptable carrier or diluent. Thereby, the absorption to the inside of the body of glucose which is the most important monosaccharide biologically is not prevented, but fructose which is not so important as glucose nutritionally is specifically prevented from absorption. And since the eucalyptus extract in the composition of the present invention is the natural-product origin, it has high safety.

Although the action mechanization that the eucalyptus extract prevents absorption of fructose in an intestinal tract is not completely clear, it guesses as follows. That is, the transporter who exists in a small intestine epithelium cell performs absorption of monosaccharides. SGLT1 which carries out active transportation only of glucose, and GLUT5 which carries out passive transportation only of fructose exist in an intraluminal side. Since the eucalyptus extract prevents absorption of fructose specifically and absorption of glucose is not prevented at all, it is guessed that the action of SGLT1 is not prevented but the action of GLUT5 is prevented.

Other compositions of the present invention comprises an extract effective in the prevention of fructose absorption, which is extracted from the plant of the genus eucalyptus; and fructose, or polysaccharide not less than disaccharide containing fructose. Examples of the composition of the present invention include food, drugs, animal feed, additive agent for animal feed, etc. Since these compositions contain fructose, they can be taken in without spoiling flavor, and moreover, they can prevent absorption of the fructose.

It is desirable that the composition of the present invention is applied to food specially. Here, examples of food include a solid food article, the half-liquid food article of the shape of cream or jam, a gel-like food, a drink, and a food additive added to these.

The composition of the present invention for preventing fructose absorption is effective in inhibition, improvement, prevention and medical treatment of hyperlipemia, fatty liver, liver cirrhosis, diabetes, high blood pressure, thrombus, circulatory organ system diseases, such as cerebral infarction and myocardial infarction, hyperuricemia, obesity, fat storage disease, arteriosclerosis etc., or inhibition or reduction of the amount of triglyceride, and the amount of cholesterol in blood.

Especially the extract in the present invention is useful for inhibition, improvement, prevention, and medical treatment of hyperlipemia and fatty liver. That is, the composition of the present invention for preventing or treating hyperlipemia and fatty liver contains, as an active ingredient, an extract effective in prevention of fructose absorption, which is extracted from the plant of the genus eucalyptus.

A composition for inhibition or amelioration accumulation of the internal-organs fat in the present invention contains, as an active ingredient, an extract effective in absorption prevention of fructose extracted from the plant of the genus eucalyptus.

### BRIEF EXPLANATION OF THE DRAWINGS

FIG. 1 is a graph showing the triglyceride fall action in blood in Examination Example 4.

### DETAILED EXPLANATION OF INVENTION

Examples of the plant of the genus eucalyptus used in the present invention include Eucalyptus globulus, Eucalyptus robusta, Eucalyptus grandis, Eucalyptus macrocarpa, Eucalyptus amygdalina, Eucalyptus macarthurii, Eucalyptus bakeri, Eucalyptus smithii, Eucalyptus microcarpa, Eucalyptus dives, Eucalyptus ovata, Eucalyptus cypellocarpa, Eucalyptus piperita, Eucalyptus polybractea, Eucalyptus wandoo, Eucalyptus citriodora, Eucalyptus viminalis, Eucalyptus botryoides, Eucalyptus calophylla, Eucalyptus salubris, Eucalyptus camaldulensis, Eucalyptus diversicolor, Eucalyptus rudis, Eucalyptus saligna, Eucalyptus coccifera, Eucalyptus deglupta, Eucalyptus racemosa, Eucalyptus oleosa, Eucalyptus paniculata, Eucalyptus delegatensis, Eucalyptus flocktoniae, Eucalyptus longifolia, Eucalyptus maculosa, Eucalyptus goniocalyx, Eucalyptus gunnii, Eucalyptus haemastoma, Eucalyptus leucoxylon, Eucalyptus perriniana, Eucalyptus melliodora, Eucalyptus nitens, Eucalyptus obliqua, Eucalyptus parvifolia, Eucalyptus pauciflora, Eucalyptus hellandra, Eucalyptus propinqua, Eucalyptus consideniana, Eucalyptus dalrympleana, Eucalyptus pulverulenta, Eucalyptus microcorys, Eucalyptus niphophila, Eucalyptus punctata, Eucalyptus radiata, Eucalyptus cinerea, Eucalyptus rubida, Eucalyptus salmonophloia, Eucalyptus dumosa, Eucalyptus ficifolia, Eucalyptus sideroxylon, Eucalyptus tereticornis, Eucalyptus regnans, Eucalyptus rostrata, Eucalyptus woolsiana, Eucalyptus blakelyi, Eucalyptus deglupta, etc., and Eucalyptus globulus or Eucalyptus robusta is desirable.

The portion into which extraction is performed among the plant of the eucalyptus genus is not restricted especially but a leaf, a seed, a bud, a trunk, a root, etc. are illustrated and a leaf is more desirable.

Extraction processing from the plant of the genus eucalyptus is performed as follows. First, a plant as a raw material, preferably leaves, is ground, and an active ingredient is extracted using water, organic solvents, or these mixtures. Examples of an organic solvent include lower alcohol (e.g. methanol, ethanol, propanol), polyhydric alcohol (e.g. glycerin, propylene glycol, diacetin, triacetin, sorbitol), ether (e.g. diethyl ether), acetone, ethyl acetate, vegetable oil fat.

As the extraction method, there is especially no restriction, and normal temperature homogenizing extraction, reflux extraction, supercritical fluid extraction, etc. can be employed.

After extraction, if needed, the resulting extract may be extracted with water saturated n-butanol, ethyl acetate, etc., and the resulting extract may be extracted again with water-containing ethanol. After extraction, the active ingredient is isolated using an adsorption chromatography, partition chromatography, an exchange chromatography, etc., and purification may be further performed according to a conventional method.

Since the Eucalyptus extract in the present invention is obtained by the treatment for extraction form the plant of the genus Eucalyptus, it exhibits sufficient safety even if it is not sufficiently fractionated and purified. To the contrary, a crude drug effect with an undetected ingredient can be expected even if it is not purified.

The Eucalyptus extract in the present invention also contains gallic acid, ellagic acid, isoquercitrin, tellimagrandin I, tellimagrandin II, pedunculagin, 1,2,4-tri-0-galloyl-β-D-glucose, 1,2,3,6-tetra-0-galloyl-β-D-glucose, 1,2,4,6-tetra-0-galloyl-β-D-glucose, pentagalloylglucose, 1,3-di-0-galloyl-4,6-hexahydroxydiphenoyl-β-D-glucose, and 1,3-di-0-galloyl-4,6-hexahydroxydiphenoyl-α-D-glucose. It is presumed that one or more kinds of these substances are correlated to the activity of the Eucalyptus extract in the present invention.

As an example of the composition of the present invention, the composition which comprises the above-mentioned extract and suitable carriers (e.g. carrier used for food or drugs), and the composition which comprises the above-mentioned extract and a polysaccharide not less than a disaccharide containing fructose are included. As an example of these compositions, the form of the food (food and drink, food additive) suitable for ingestion, drugs, animal feed, and additive agent for animal feed is mentioned.

Examples of the above-mentioned polysaccharide include sucrose (sugar), lactosucrose, raffinose, erulose, 1-kestose, inulin. In case the eucalyptus extract is taken in to a man or a mammal with the above-mentioned forms, it is usually desirable that the dose of this extract is the range of 0.01-300 mg/kg weight per day, but even if the dose exceeds 300 mg/kg weight per day, it is satisfactory at safety, since it is the natural product of the genus eucalyptus origin.

In order to make the composition into a food form, the eucalyptus extract is mixed with the various ingredients used for food, and is prepared by the form of a solid food article, a half-liquid food article (e.g. the shape of cream or jam), gel-like food, a drink). Moreover, the eucalyptus extract may be blended in food with the shape of remaining as it is or a powder, a granule, a capsule, a tablet, and a liquid which are manufactured by blending the suitable carrier, if needed. When the eucalyptus extract is used with the above food forms, the eucalyptus extract may be used together with fructose and/or polysaccharide containing fructose, whereby the food which excels in acceptability, and can prevent absorption of the fructose can be manufactured.

Other components blended in the food with the eucalyptus extract are not especially restricted, and each various components usually used can be used. Examples of such components include grape sugar, maltose, sorbitol, stevioside, corn syrup, lactose, citric acid, tartaric acid, malic acid, succinic acid, lactic acid, L-ascorbic acid, dl-α-tocopherol, glycerol, propylene glycol, glycerine fatty acid ester, poly glycerine fatty acid ester, cane sugar fatty acid ester, sorbitan fatty acid ester, propylene-glycol fatty acid ester, gum arabic, carrageenan, casein, gelatin, pectin, agar, vitamin B group, nicotinamide, calcium pantothenate, amino acids, calcium salts, coloring matter, perfume and a preservation agent, and these components may be suitably blended according to the kind of food.

Examples of the food include soft drink, juice, coffee, tea, liqueur, cow's milk, milk serum drink, lactic acid bacteria beverage, candy, chewing gum, chocolate, gumdrop, yogurt, ice cream and pudding. The amount of the Eucalyptus extract contained in food is preferably within a range from 0.5 to 100 mg/g. Even if the Eucalyptus extract is incorporated in the amount which exceeds the above range, the safety and effect are not impaired.

When using the eucalyptus extract as a food additive, it may be added in food as it is, or may be added after mixing with fructose or polysaccharide not less than disaccharide containing fructose. The form of the food additive may include powder, granule, capsule, sirup, gel, liquid and solid. The food that adds this food additive is not especially restricted, and various cooking foods and processed foods are included. The amount of addition of the eucalyptus extract may be of the same grade as the amount of the food. Any stage before cooking during cooking and after cooking is suitable as the addition time of a food additive.

In order to use the composition of the present invention in the form of drugs, the Eucalyptus extract is formed into the form of a solid, semi-solid or liquid by adding a conventional biologically acceptable carrier or diluent. Specific form includes, for example, oral agents such as tablets, capsules, pills, granules, powders, emulsions, suspensions, syrups, and pellets; and parenteral agents such as injections, drops, and suppositories

In case of forming into a preparation, there can be used carriers which are normally used according to dosage forms, for example, surfactants, excipients, binders, disintegrators, lubricants, preservatives, stabilizers, buffers, and suspensions. Preferred examples thereof include solid carriers such as starch, lactose, mannitol, carboxymethylcellulose, corn starch, and inorganic salt; liquid carriers (diluents) such as distilled water, saline, aqueous glucose solution, alcohol (e.g. ethanol), propylene glycol, and polyethylene glycol; and oily carriers such as various animal and vegetable oils, white soft paraffin, paraffin and wax.

Examples of the drugs include an anti-obesity agent, an inhibitor of accumulation of fat (e.g. offal fat and subcutaneous fat), an anti-arteriosclerosis agent, a thrombus inhibitor, a triglyceride fall agent and the cholesterol fall agent in blood, besides agent of prevention or treatment of hyperlipidemia and fatty liver.

The animal feed in the present invention is prepared by mixing the Eucalyptus extract with various components used in the animal feed. Specific examples of the animal feed include feed for livestock, and pet foods such as cat food and dog food. The amount of the Eucalyptus extract contained in the animal feed is preferably within a range from 0.5 to 100 mg/g. Even if the Eucalyptus extract is incorporated in the amount which exceeds the above range, the safety and effect are not impaired.

The form of the additive for the animal feed is not specifically limited and the Eucalyptus extract may be added to animal feed as it is, or the Eucalyptus extract may also be prepared in the form of a powder, capsule, syrup, gel, liquid or solid. The animal feed as described above is mentioned as an example of the animal feed which adds the additive for animal feed. It is suitable that the amount of the Eucalyptus extract to be added to the additive is of the same grade as the amount of blending in the animal feed. Which stage during and after manufacture of animal feed is suitable as the addition stage of the additive to animal feed.

### EXAMPLE

Hereafter, the present invention is explained in detail using the reference example and the test examples.

### Reference Example

Dried leaves (500 g) of Eucalyptus globulus were refluxed with 4.5 kg of 33% ethanol for two hours. After the resulting solution was cooled to room temperature and filtrated, the filtrate was allowed to stand in a refrigerator overnight. After filtrate was further filtrated, the resulting filtrate was concentrated under reduced pressure and freeze-dried to obtain a Eucalyptus extract.

### Test Example 1

In order to confirm the safety of the eucalyptus extract, the following tests were conducted using the eucalyptus extract obtained in the Reference Example.

(1) Acute Toxicity Test

The acute toxicity test by oral administration was conducted using Wistar SPF rats. That is, the 2 g eucalyptus extract per weight of 1 kg was administered orally using a stomach sonde to the rats, and action of rats was observed for 24 hours (n=5). Then, the rats were slaughtered and dissected, and it confirmed whether each organization, such as internal organs, would have neither a pathological change nor malformation.

As a result, abnormalities were not observed in action of each rat after 24-hour progress at all, and abnormalities, such as malformation, were not observed by each organization in the inspection by dissection.

(2) Subacute Toxicity Test

The subacute toxicity test by oral administration was conducted using Wistar SPF rats. That is, the rats were divided into the control group (n=7) to which the athrocytosis of the usual feed is carried out, and the eucalyptus group (n=7) to which the athrocytosis of the feed which mixed the eucalyptus extract of 1 volume % with usual feed is carried out, and bred each individually. The rats of these groups were made to carry out free ingestion of feed and the potable water. The usual feed used in this test does not contain fructose.

The amount of food ingested and the amount of drinking water of rats of these groups were measured every day, and weight change was measured twice per week. The test was continued for five weeks. After the test end, by collecting blood from the heart, rats were made to die and pathological changes, malformation, etc. of each organization, such as internal organs, were confirmed after dissection.

As a result, in the amount of food ingested, the amount of drinking water, and weight change, the significant difference was not accepted between the control group and the eucalyptus group. Moreover, abnormalities, such as malformation, were not observed by each organization in inspection by dissection. The significant difference was not observed when the amount of cholesterol and the amount of triglyceride in liver were measured. The difference significant in the blood sugar level, the amount of triglyceride in blood, and the total amount of cholesterol in blood was not observed.

### Test Example 2

The sugar load test to fructose was conducted using the eucalyptus extract obtained in the Reference Example. First, Wistar SPF rats (male) were divided into the eucalyptus group with which the eucalyptus extract is dosed, and the control group with which the eucalyptus extract is not dosed, and the rats of each group were made to abstain from food for 18 hours. Subsequently, the rats of the eucalyptus group were dosed with the test solution prepared so that the dose of the eucalyptus extract might be set to 1 g per weight of 1 kg by a stomach sonde. After 10 minutes, the rats of the eucalyptus group and control group were dosed with the test solution prepared so that the dose of a fructose might be set to 1 g per weight of 1 kg by a stomach sonde. Blood was collected from the portal vein, respectively just before dosing of fructose, after 30 minutes of dosing, and after 60 minutes of dosing, and the fructose concentration in portal vein blood was measured by the following methods.

Fructose concentration of portal vein blood was measured by the enzymatic process which uses D-fructose dehydrogenase (Gluconobacter sp. origin). That is, after adding blood serum 0.01 ml to 0.99 ml of a mixed-solution which contains 100 mM phosphoric acid buffer (pH 6.0), 1% Triton X-100, 0.2 mM WST-1, 8 μM 1-methoxy PMS, and 5U fructose dehydrogenase, and making it react at 30° C. for 3 hours. The amount of fructose was determined by measuring 438 nm absorption of generated reduction-type WST-1. Each conditions was examined by n=5. The measurement result is shown in Table 1.

TABLE 1

| | |
|---|---|
| Control group | Eucalyptus group |

Fructose concentration
in portal vein blood (mM)

| | | |
|---|---|---|
| 0 minute | 0.02 ± 0.005 | — |
| | (0.4 ± 0.08) | |
| 30 minutes | 1.11 ± 0.368 | 0.15 ± 0.044* |
| | (19.9 ± 6.62) | (2.6 ± 0.79) |
| 60 minutes | 0.79 ± 0.303 | 0.24 ± 0.048* |
| | (14.3 ± 5.46) | (4.3 ± 0.87) |

Unit of the numerical value in a parenthesis: mg/dL
*P < 0.05

Table 1 shows the average of fructose concentration in the portal vein blood just before fructose dosing (0 minute), after 30 minutes of dose and after 60 minutes of dose. As is apparent from Table 1, the increase of fructose concentration in the eucalyptus group wherein the eucalyptus extract obtained in the Reference Example was dosed to rats is intentionally inhibited as compared with the control group. This shows that the eucalyptus extract has prevented absorption of fructose in an intestinal tract.

### Test Example 3

The sugar load test to glucose was conducted using the eucalyptus extract obtained in the Reference Example. That is, Wistar SPF rats (male) were divided into the eucalyptus group with which the eucalyptus extract is dosed, and the control group with which the eucalyptus extract is not dosed, and the rats of each group were made to abstain from food for 18 hours. Subsequently, the rats of a eucalyptus group were dosed with the test solution prepared so that the dose of the eucalyptus extract might be set to 1 g per weight of 1 kg by the stomach sonde. After 10 minutes, the rats of the eucalyptus group and the control group were dosed with the test solution prepared so that the dose of glucose might be set to 1 g per weight of 1 kg by the stomach sonde.

Blood was collected from the portal vein just before glucose dosing and after 30 minutes of dosing, respectively, and glucose concentration in portal vein blood was measured by the “new blood sugar test” of Boehringer Mannheim company. Each conditions examined by n=5. The measurement result is shown in Table 2.

TABLE 2

| | |
|---|---|
| Control group | Eucalyptus group |

Glucose concentration
in portal vein blood (mM)

| | | |
|---|---|---|
| 0 minute | 9.6 ± 1.25 | — |
| | (172 ± 22.5) | |
| 30 minutes | 17.3 ± 2.46 | 16.5 ± 2.39 |
| | (311 ± 44.2) | (298 ± 43.0) |

Unit of the numerical value in a parenthesis: mg/dL

Table 2 shows the average of glucose concentration in the portal vein blood just before glucose dosing (0 minute) and after 30 minutes from dosing. As is apparent from Table 2, in glucose concentration, a significant difference is not observed between the eucalyptus group which dosed the rat with the eucalyptus extract obtained in the Reference Example, and the control group. This shows that the eucalyptus extract has not prevented absorption of glucose.

### Test Example 4

(1) Long-term Breeding of Rats

As shown below, long-term breeding of the rats was carried out using the eucalyptus extract obtained in the Reference Example.

First, seven Wistar SPF rats (male) were divided into each two groups after one-week preliminary breeding, and free ingestion of the test meals (high fructose foods) of compositions shown in Table 3 was carried out at each group for five weeks. Breeding was conducted on conditions of the temperature of 23±2° C., 55±5% of humidity, and 12 hours/day of lighting. The group which took in the test meal by which the eucalyptus extract was added is called a eucalyptus group, and the group which took in the test meal by which the eucalyptus extract was not added is called a control group.

TABLE 3

The amount of blending (g)

| Component | Control group | Eucalyptus group |
|---|---|---|
| Casein | 170 | 170 |
| Fructose | 700 | 700 |
| Vitamins | 10 | 10 |
| Minerals | 40 | 40 |
| Choline chloride | 2 | 2 |
| Methionine | 3 | 3 |
| Soybean oil | 30 | 30 |
| Crystalline cellulose | 45 | 35 |
| Eucalyptus extract | 0 | 10 |

(2) Test for Measuring the Amount of Triglyceride in the Liver

A liver was extracted after the breeding end of the above (1). After treating this liver by the conventional method, the amount of triglyceride in the liver was measured by “Triglyceride G-Test Wako” manufactured by Wako Pure Chemical industries, Ltd. The result is shown in Table 4.

As is apparent from Table 4, triglyceride accumulation in the liver in the eucalyptus group was inhibited as Compared with the control group. This result shows that dosing of a eucalyptus extract is effective in inhibition, amelioration, prevention and treatment of fatty liver and liver cirrhosis resulting from accumulation of the triglyceride in liver.

TABLE 4

| | Control group | Eucalyptus group |
|---|---|---|
| Amount of triglyceride | 37.0 ± 6.66 | 13.8 ± 4.30* |
| in liver (mg/g liver) | | |

*P < 0.05

(3) Test for Measuring the Amount of Triglyceride in Blood

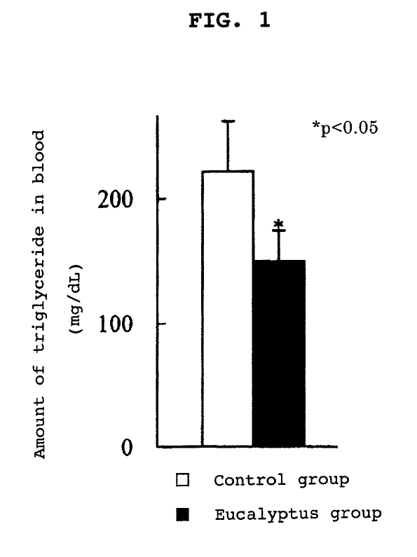

After the breeding end of the above (1), cardiac blood collecting was carried out and the amount of triglyceride in blood was measured by the same manner as the above test (2) after trating by the conventional method. The result is shown in FIG. 1. As is apparent from FIG. 1, the amount of triglyceride in blood in the eucalyptus group was reduced as compared with the control group. This result shows that administration of the eucalyptus extract is effective in inhibition, amelioration, prevention and medical treatment of the diseases which originate in that there are many amounts of triglyceride in blood, for example, hyperlipidemia, arteriosclerosis, hypercholesterolemia, a thrombus and the like.

What is claimed is:

1. A composition for preventing fructose absorption, comprising:
(1) a biologically effective amount of an extract of a plant of the genus Eucalyptus as an active ingredient, and
(2) a biologically acceptable carrier or diluent.

2. A composition according to claim 1 wherein at least one selected from fructose and polysaccharide not less than disaccharide containing fructose is further contained.

3. A composition comprising:
(1) a biologically effective amount of an extract of a plant of the genus Eucalyptus as an active ingredient, and
(2) at least one selected from fructose and polysaccharide not less than disaccharide containing fructose.

4. A Composition according to claim 3 wherein polysaccharide is sucrose.

5. A composition for prevention or treatment of hyperlipidemia, comprising:
(1) a biologically effective amount of an extract of a plant of the genus Eucalyptus as an active ingredient, and
(2) a biologically acceptable carrier or diluent.

6. A composition for prevention or treatment of fatty liver, comprising:
(1) a biologically effective amount of an extract of a plant of the genus Eucalyptus as an active ingredient, and
(2) a biologically acceptable carrier or diluent.

7. A composition for inhibition or amelioration of accumulation of internal-organs fat, comprising:
(1) a biologically effective amount of an extract of a plant of the genus Eucalyptus as an active ingredient, and
(2) a biologically acceptable carrier or diluent.

8. A method of preventing fructose absorption, which comprises administering or ingesting a composition comprising:
(1) a biologically effective amount of an extract of a plant of the genus Eucalyptus as an active ingredient, and
(2) a biologically acceptable carrier or diluent.

9. A method according to claim 8 wherein the composition contains at least one selected from fructose and polysaccharide not less than disaccharide containing fructose

10. A method of preventing or treating fatty liver, which comprises administering or ingesting a composition comprising:
(1) a biologically effective amount of an extract of a plant of the genus Eucalyptus as an active ingredient, and
(2) a biologically acceptable carrier or diluent.
